SEQUENCE LISTING (22 sequences)

SEQ ID NO: 1 - DNA, 4138 bp, Plasmodium falciparum
tttataatatcgtttttttatgagcattatatcatataaataattaattaaataaatatatatatatatataattataattagaccaataaattatatataatgaaaggatattttaatatatattttttaattcctttaatttttttatataatgtaataagaataaatgaatcaataataggtagaacactttataatagacaagatgaatcatcagatatttcaagggtaaattcacccgaattaaataataatcataaaactaatatatatgattcagattacgaagatgtaaataataaattaataaacagttttgtagaaaataaaagtgtgaaaaaaaaaaggtctttaagttttataaataataaaacaaaatcatatgatataattccaccttcatattcatataggaatgataaatttaattcactttccgaaaatgaagataattctggaaatacaaatagtaataatttcgcaaatacttctgaaatatctattggaaaggataataaacaatatacgtttatacagaaacgtactcatttgtttgcttgtggaataaaaagaaaatcaataaaatggatatgtcgagaaaacagtgagaaaattactgtatgtgttcctgatagaaaaatacaactatgtgttgcaaattttttaaactcacgtttagaaacaatggaaaagtttaaagaaatatttttaatttctgttaatacagaagcaaaattattatataacaaaaatgaaggaaaagatccctcaatattttgtaatgaattaagaaatagtttttcagattttagaagttcatttataggtgatgatatggattttggtggtaatacagatagagtcaaaggatatattaatacgaagttctccgattattataaggaaaaaaatgttgaaaaattaaataatatcaaaaaagaatggtgggaaaaaaataaagcaaatttgtggaatcacatgatagtaaatcataaaggaaacataagtaaagaatgtgccataattcccgcggaagaacctcaaattaatctatggataaaagaatggaatgaaaacttcttgatggaaaagaagagattgtttttaaatataaaagataagtgtgttgaaaacaaaaaatatgaagcatgttttggtggatgtaggcttccatgttcttcatatacatcatttatgaaaaaaagtaaaacacaaatggaggttttgacgaacttgtataaaaagaaaaattcaggagtggataaaaataattttctgaatgatctttttaaaaaaaataataaaaatgatttagatgattttttcaaaaatgaaaaggaatatgatgatttatgtgattgcagatatactgctactattattaaaagttttctaaatggtcctgctaaaaatgatgtagatattgcatcacaaattaatgttaatgatcttcgagggtttggatgtaattataaaagtaataatgaaaaaagttggaattgtactggaacatttacgaacaaatttcctggtacatgtgaaccccccagaagacaaactttatgtcttggacgtacatatcttttacatcgtggtcatgaggaagattataaggaacatttacttggagcttcaatatatgaggcgcaattattaaaatataaatataaggaaaaggatgaaaatgcattgtgtagtataatacaaaatagttatgcagatttggcagatattatcaagggatcggatataataaaagattattatggtaaaaaaatggaagaaaatttaaataaagtaaacaaagataaaaaacgtaatgaagaatctttgaagatttttcgtgaaaaatggtgggatgaaaacaaggagaatgtatggaaagtaatgtcagcagtacttaaaaataaggaaacgtgtaaagattatgataagtttcaaaagattcctcaatttttaagatggtttaaggaatggggagacgatttttgtgagaaaagaaaagagaaaatatattcatttgagtcatttaaggtagaatgtaagaaaaaagattgtgatgaaaatacatgtaaaaataaatgtagtgaatataaaaaatggatagatttgaaaaaaagtgaatatgagaaacaagttgataaatacacaaaagataaaaataaaaagatgtatgataatattgatgaagtaaaaaataaagaagccaatgtttacttaaaagaaaaatccaaagaatgtaaagatgtaaatttcgatgataaaatttttaatgagagtccaaatgaatatgaagatatgtgtaaaaaatgtgatgaaataaaatatttaaatgaaattaaatatcctaaaacaaaacacgatatatatgatatagatacattttcagatacttttggtgatggaacgccaataagtattaatgcaaatataaatgaacaacaaagtgggaaggatacctcaaatactggaaatagtgaaacatcagattcaccggttagtcatgaaccagaaagtgatgctgcaattaatgtagaaaagttaagtggtgatgaaagttcaagtgaaacaagaggaatattagatattaatgatccaagtgttacgaacaatgtcaatgaagttcatgatgcttcaaatacacaaggtagtgtttcaaatacttctgatataacgaatggacattcggaaagttccctgaatagaacaacgaatgcacaagatattaaaataggccgttcaggaaatgaacaaagtgataatcaagaaaatagttcacattctagtgataattcaggttctttgacaatcggacaagttccttcagaggataatacccaaaatacatatgattcacaaaaccctcatagagatacacctaatgcattagcatctttaccatcagatgataaaattaatgaaatagagggtttcgattctagtagagatagtgaaaatggtaggggtgatacaacatcaaatactcatgatgtacgtcgtacgaatatagtaagtgagagacgtgtgaatagccatgattttattagaaacggaatggcgaataacaatgcacatcatcaatatataacgcaaattgagaataatggaatcataagaggacaagaggaaagtgcggggaatagtgttaattataaagataatccaaagaggagtaatttttcctccgaaaatgatcataagaaaaatatacaggaatataattctagagatactaaaagagtaagggaggaaataattaaattatcgaagcaaaataaatgcaacaatgaatattccatggaatattgtacctattctgacgaaaggaatagttcaccgggtccttgttctagagaagaaagaaagaaattatgttgtcagatttcagattattgtttaaaatattttaacttttattcaattgaatattataattgtataaaatctgaaattaaaagtccagaatataaatgttttaaaagcgagggtcaatcaagtatgtttcatatataatgaaaataactaaattaaaataaatatctaaattttccatttaaatataatttaaataatttatttttttattataaaatttatttgtttttttttatataatgttttattttttctttacaggcattccttattttgctgctggaggtattttagttgtaatagtcttacttttgagttcagcatctagaatggggaaaaggttacacgcataagattaaataaatatgactatactttttaatttttatattatttatattattattatgttttaacaagaatataaattatattatatatttatatgtatatatatttttttatatagtaatgaagaatatgatataggagaatctaatatagaagcaacttttgaagaaaataattatttaaataaactatcgcgcatatgtaagattaatataaaaagtgaaaatttcatattaaataaataaatgaatatatatatatatatatatatatatatatatatatatacatgttatataaattttaatgttatattatttacttttttctcagttaatcaagaagtacaagagacaaacatttcagattattccgagtacaattataatgaaaagaatatgtattaattt

SEQ ID NO: 2 - protein, 1210 aa, Plasmodium falciparum
MKGYFNIYFLIPLIFLYNVIRINESIIGRTLYNRQDESSDISRVNSPELNNNHKTNIYDSDYEDVNNKLINSFVENKSVKKKRSLSFINNKTKSYDIIPPSYSYRNDKFNSLSENEDNSGNTNSNNFANTSEISIGKDNKQYTFIQKRTHLFACGIKRKSIKWICRENSEKITVCVPDRKIQLCVANFLNSRLETMEKFKEIFLISVNTEAKLLYNKNEGKDPSIFCNELRNSFSDFRSSFIGDDMDFGGNTDRVKGYINTKFSDYYKEKNVEKLNNIKKEWWEKNKANLWNHMIVNHKGNISKECAIIPAEEPQINLWIKEWNENFLMEKKRLFLNIKDKCVENKKYEACFGGCRLPCSSYTSFMKKSKTQMEVLTNLYKKKNSGVDKNNFLNDLFKKNNKNDLDDFFKNEKEYDDLCDCRYTATIIKSFLNGPAKNDVDIASQINVNDLRGFGCNYKSNNEKSWNCTGTFTNKFPGTCEPPRRQTLCLGRTYLLHRGHEEDYKEHLLGASIYEAQLLKYKYKEKDENALCSIIQNSYADLADIIKGSDIIKDYYGKKMEENLNKVNKDKKRNEESLKIFREKWWDENKENVWKVMSAVLKNKETCKDYDKFQKIPQFLRWFKEWGDDFCEKRKEKIYSFESFKVECKKKDCDENTCKNKCSEYKKWIDLKKSEYEKQVDKYTKDKNKKMYDNIDEVKNKEANVYLKEKSKECKDVNFDDKIFNESPNEYEDMCKKCDEIKYLNEIKYPKTKHDIYDIDTFSDTFGDGTPISINANINEQQSGKDTSNTGNSETSDSPVSHEPESDAAINVEKLSGDESSSETRGILDINDPSVTNNVNEVHDASNTQGSVSNTSDITNGHSESSLNRTTNAQDIKIGRSGNEQSDNQENSSHSSDNSGSLTIGQVPSEDNTQNTYDSQNPHRDTPNALASLPSDDKINEIEGFDSSRDSENGRGDTTSNTHDVRRTNIVSERRVNSHDFIRNGMANNNAHHQYITQIENNGIIRGQEESAGNSVNYKDNPKRSNFSSENDHKKNIQEYNSRDTKRVREEIIKLSKQNKCNNEYSMEYCTYSDERNSSPGPCSREERKKLCCQISDYCLKYFNFYSIEYYNCIKSEIKSPEYKCFKSEGQSSIPYFAAGGILVVIVLLLSSASRMGKSNEEYDIGESNIEATFEENNYLNKLSRIFNQEVQETNISDYSEYNYNEKNMY

SEQ ID NO: 3 - DNA, 29 bp, Artificial Sequence
  primer
agaccaataaattatatataatgaaagga

SEQ ID NO: 4 - DNA, 27 bp, Artificial Sequence
  primer
tttaaacttttccattgtttctaaacg

SEQ ID NO: 5 - DNA, 28 bp, Artificial Sequence
  primer
ataaatttaattcactttccgaaaatga

SEQ ID NO: 6 - DNA, 26 bp, Artificial Sequence
  primer
aaaacaatctcttcttttccatcaag

SEQ ID NO: 7 - DNA, 26 bp, Artificial Sequence
  primer
tttataggtgatgatatggattttgg

SEQ ID NO: 8 - DNA, 25 bp, Artificial Sequence
  primer
tcgtaaatgttccagtacaattcct

SEQ ID NO: 9 - DNA, 24 bp, Artificial Sequence
  primer
caaatggaggttttgacgaacttg

SEQ ID NO: 10 - DNA, 25 bp, Artificial Sequence
  primer
taagtactgctgacattactttcca

SEQ ID NO: 11 - DNA, 23 bp, Artificial Sequence
  primer
ggagcttcaatatatgaggcgca

SEQ ID NO: 12 - DNA, 26 bp, Artificial Sequence
  primer
atatcttcatattcatttggactctc

SEQ ID NO: 13 - DNA, 26 bp, Artificial Sequence
  primer
tgagtcatttaaggtagaatgtaaga

SEQ ID NO: 14 - DNA, 24 bp, Artificial Sequence
  primer
ggaactttccgaatgtccattcgt

SEQ ID NO: 15 - DNA, 25 bp, Artificial Sequence
  primer
taaatgaacaacaaagtgggaagga

SEQ ID NO: 16 - DNA, 27 bp, Artificial Sequence
  primer
attctcaatttgcgttatatattgatg

SEQ ID NO: 17 - DNA, 24 bp, Artificial Sequence
  primer
agttccttcagaggataataccca

SEQ ID NO: 18 - DNA, 25 bp, Artificial Sequence
  primer
cttgattgaccctcgcttttaaaac

SEQ ID NO: 19 - DNA, 26 bp, Artificial Sequence
  primer
actaaaagagtaagggaggaaataat

SEQ ID NO: 20 - DNA, 31 bp, Artificial Sequence
  primer
tataaaatacattgaattatttaaactattg

SEQ ID NO: 21 - DNA, 24 bp, Artificial Sequence
  oligonucleotide
attccttattttgctgctggaggt

SEQ ID NO: 22 - DNA, 26 bp, Artificial Sequence
  oligonucleotide
aagttgcttctatattagattctcct

(19) United States
(12) Patent Application Publication
(10) Pub. No.: US 2005/0239730 A1
(43) Pub. Date: Oct. 27, 2005

(54) Plasmodium falciparum erythrocyte binding protein baebl for use as a vaccine

(76) Inventors: Ghislaine Mayer, Gaithersburg, MD (US); Louis H. Miller, Rockville, MD (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/677,980

(22) Filed: Oct. 2, 2003

Related U.S. Application Data
(63) Continuation of application No. PCT/US02/10071, filed on Mar. 29, 2002.
(60) Provisional application No. 60/281,130, filed on Apr. 2, 2001.

(51) Int. Cl. A61K 48/00; A61K 39/002

(52) U.S. Cl. 514/44; 424/191.1

(57) ABSTRACT

The invention relates to Plasmodium falciparum Erythrocyte Binding Protein BAEBL for use as a vaccine.

### RELATED APPLICATIONS

This application is a continuation and claims the benefit of priority of International Application No. PCT/US02/10071 filed Mar. 29, 2002, designating the United States of America and published in English, which claims the benefit of priority of U.S. Provisional Application No. 60/281,130 filed Apr. 2, 2001, both of which are hereby expressly incorporated by reference in their entireties.

### FIELD OF THE INVENTION

The invention relates to Plasmodium falciparum Erythrocyte Binding Protein BAEBL for use as a vaccine.

### BACKGROUND OF THE INVENTION

The erythrocytic stage of Plasmodium falciparum causes several million deaths yearly, primarily in Africa. The parasite lives within the erythrocyte except during the brief period when merozoites, the invasive stage of the parasite, are released from infected erythrocytes to invade uninfected erythrocytes. Invasion of erythrocytes by merozoites is a multistep process that includes: attachment, reorientation of the merozoite in such a way that its apical end is in contact with the erythrocyte surface, junction formation, and entry into the parasitophorous vacuole (Dvorak, J. A. et al. 1975 Science 187: 748-9; Aikawa, M. et al. 1978 J Cell Biol 77: 72-82). The binding of merozoites to erythrocytes requires parasite receptors (Camus, D. & Hadley, T. H. 1985 Science 230: 553-556; Haynes, J. D. et al. 1988 J Exp Med 167: 1873-1881; Adams, J. H. et al. 1990 Cell 63: 142-153; Sim, B. K. L. et al. 1990 J Cell Biol 111: 1877-1884; Galinski, M. R. et al. 1992 Cell 69: 1213-1226).

One family of these parasite receptors is named Duffy binding-like erythrocyte binding protein (DBL-EBP) for its similarity to the P. vivax and P. knowlesi proteins that bind to the Duffy blood group antigens (Duffy positive) on human erythrocytes (Adams, J. H. et al. 1992 PNAS USA 89: 7085-7089). P. vivax does not infect Africans lacking the Duffy blood group antigens (Duffy negative), and P. knowlesi will not form a junction with or invade Duffy negative human erythrocytes (Miller, L. H. et al. 1976 N Engl J Med 295: 302-304; Miller, L. H., et al. 1979 J Exp Med 149: 172-184). Region II, a domain of the P. vivax DBL-EBP, has the same specificity as the full-length protein (Chitnis, C. E. & Miller, L. H. 1994 J Exp Med 180: 497-506).

P. knowlesi has three highly homologous DBL proteins, each with different specificities as defined by region II (Haynes, J. D. et al. 1988 J Exp Med 167: 1873-1881; Ranjan, A. & Chitnis, C. E. 1999 PNAS USA 96: 14067-14072). One binds to Duffy blood group antigens on human and rhesus erythrocytes, a second binds to sialic acid on rhesus erythrocytes, and a third binds to an unidentified receptor on rhesus erythrocytes. Whereas P. knowlesi can only invade Duffy positive human erythrocytes, it can invade rhesus erythrocytes that have been rendered Duffy negative by protease treatment and by removal of sialic acid with neuraminidase (Haynes, J. D. et al. 1988 J Exp Med 167: 1873-1881; Miller, L. H. et al. 1973 J Exp Med 138: 1597-1601). P. knowlesi invades these enzymatically treated erythrocytes at the same rate as the untreated erythrocytes, indicating a highly efficient alternative pathway of invasion.

The Duffy binding proteins of P. vivax (PvDBP) and P. knowlesi (PkDBP) are part of a larger family of Plasmodium proteins that include EBA-175 of P. falciparum. EBA-175 binds to sialic acid and the peptide backbone of glycophorin A on the erythrocyte surface (Sim, B. K. L. et al. 1994 Science 264: 1941-1944). As in the case of P. vivax, the binding domain of EBA-175 is defined by region II. Unlike P. vivax, which cannot infect Duffy negative erythrocytes, some strains of P. falciparum parasites have alternative pathways of invasion, not requiring glycophorin A for either invasion or growth in vitro. Thus, other receptors must be involved in these alternative pathways (Dolan, S. A. et al. 1990 J Clin Invest 86: 618-624).

The P. falciparum genome sequence identifies at least four paralogues of EBA-175. We have studied one of these DBL genes of P. falciparum, named baebl (Adams, J. H. et al. 2001 Trends Parasitol 17: 297-9), to explore its possible role in invasion.

### SUMMARY OF THE INVENTION

The present invention relates to Plasmodium falciparum Erythrocyte Binding Protein BAEBL for use as a vaccine. A BAEBL polynucleotide sequence or a portion thereof, or a BAEBL polypeptide sequence or a portion thereof, is used to induce an immune response to a Plasmodium parasite, whereby a human is protected against malaria. The BAEBL polynucleotide sequence is alternatively used to express recombinant polypeptides or portions thereof. Furthermore, synthetic BAEBL polypeptides or portions thereof are prepared in accordance with the present invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1. Sequencing strategy and exon/intron structure of baebl. Oligonucleotides were designed based on the genomic sequence obtained from the Plasmodium falciparum genome project (Sanger Centre) and used for sequencing of genomic DNA (GenBank No. AF332918) and RT-PCR of mRNA (GenBank No. AF332919) to determine the intron/exon structure in P. falciparum Dd2/Nm strain. (A) Schematic representation of the gene and predicted protein structure of baebl. Predicted protein structure has strong similarity with EBA-175, containing the putative signal sequence (SS; aa 1-21) predicted by SIGNALP V2.0; region II (two Duffy binding-like (DBL) domains, F1 and F2); region VI (3° Cys), the transmembrane domain (TM; aa 1134-1153) predicted by TMHMM V2.0, followed by the putative cytoplasmic domain (Cyt). (B) f1 to f9 primers (see Examples) are used for RT-PCR of mRNA (lanes marked c) and PCR of genomic DNA (lanes marked g). Bar=1 kb.

FIG. 2. Confocal microscopy demonstrates the localization of BAEBL in micronemes. (A) Dd2/Nm schizonts were double labeled with anti-BAEBL region II and anti-EBA-175. Schizonts immunolabeled with anti-BAEBL region II were stained with Alexa 488 secondary antibody. Schizonts labeled with anti-EBA-175 were stained with Alexa 594 secondary antibody. (B) Dd2/Nm schizonts were double labeled with anti-BAEBL region VI and anti-EBA-175. Schizonts immunolabeled anti-BAEBL region VI were stained with Alexa 488 secondary antibody. Schizonts labeled with anti-EBA-175 were stained with Alexa 594 secondary antibody. (C) Dd2/Nm schizonts were double labeled with anti-BAEBL region II and anti-RAP-1 monoclonal antibody. Schizonts immunolabeled anti-BAEBL region II were stained with Alexa 488 secondary antibody. Schizonts labeled with anti-RAP-1 were stained with TRITC secondary antibody. (D) Dd2/Nm schizonts were double labeled with anti-BAEBL region VI and anti-RAP-1 monoclonal antibody. Schizonts immunolabeled with anti-BAEBL region VI were stained with Alexa 488 secondary antibody. Schizonts labeled with anti-RAP-1 were stained with TRITC secondary antibody.

FIG. 3. Evidence that anti-region II (Anti-R2) and anti-region VI (Anti-R6) sera immunoprecipitate the same protein. The supernatant was preabsorpted with either anti-region 2 or anti-region 6 followed by immunoprecipitation by the two sera. BAEBL was removed by both sera.

FIG. 4. BAEBL and EBA-175 did not bind to neuraminidase (NM eluate) or trypsin-treated erythrocytes (Trypsin RBC). Eluates of BAEBL and EBA-175 were only seen from normal erythrocytes.

FIG. 5. BAEBL binds and is eluted from En(a-) erythrocytes that lack glycophorin A. NM RBC are neuraminidase-treated normal erythrocytes.

FIG. 6. Absorption and elution of BAEBL (A, B) and EBA-175 (C, D) with various amounts (25, 50, 2×50 μl, and 4×50 μl of packed erythrocytes) of Gerbich [Ge(-2, -3, 4)], normal, and neuraminidase (NM)-treated erythrocytes. For elution, 25 μl and 50 μl of packed erythrocytes were used.

### SUMMARY OF SEQUENCES

SEQ ID NO: 1 and FIG. 7 are the genomic sequence encoding Plasmodium falciparum Erythrocyte Binding Protein BAEBL; start and stop codons are indicated in bold, and the introns span nucleotides 3499-3638, 3718-3846, and 3930-4061.

SEQ ID NO: 2 is the amino acid sequence encoding Plasmodium falciparum Erythrocyte Binding Protein BAEBL.

### DETAILED DESCRIPTION OF THE PREFERRED EMBODIMENT

A member of a Plasmodium receptor family for erythrocyte invasion was identified on chromosome 13 from the Plasmodium falciparum genome sequence of the Sanger Centre. The protein (named BAEBL) has homology to EBA-175, a P. falciparum receptor that binds specifically to sialic acid and the peptide backbone of glycophorin A on erythrocytes. Both EBA-175 and BAEBL localize to the micronemes, an organelle at the invasive end of the parasite that contains other members of the family. Like EBA-175, the erythrocyte receptor for BAEBL is destroyed by neuraminidase and trypsin, indicating that the erythrocyte receptor is a sialoglycoprotein. Its specificity, however, differs from EBA-175 in that BAEBL can bind to erythrocytes that lack glycophorin A, the receptor for EBA-175. It has reduced binding to erythrocytes with the Gerbich mutation found in another erythrocyte sialoglycoprotein (glycophorin C/D). The interest in BAEBL's reduced binding to Gerbich erythrocytes derives from the high frequency of the Gerbich phenotype in some regions of Papua New Guinea where P. falciparum is hyperendemic.

The present invention relates, in general, to a substantially purified polynucleotide sequence (e.g. a DNA sequence) encoding all, or a portion, of BAEBL of the DBL-EBP family of a Plasmodium parasite (particularly, Plasmodium falciparum). A “portion” as used herein preferably consists of at least five (or six) amino acids or, correspondingly, at least 15 (or 18) nucleotides. GenBank No. AF332918 encodes the genomic DNA and GenBank No. AF332919 encodes the cDNA of BAEBL.

The present invention further relates to a polynucleotide sequence encoding a BAEBL protein of other Plasmodium parasites such as, for example, P. vivax or P. knowlesi. One of ordinary skill in the art, given the present disclosure, could easily identify and clone analogous genes in such species without undue experimentation.

In one embodiment, the present invention relates to a polynucleotide sequence given in SEQ ID NO: 1 encoding the entire amino acid sequence of BAEBL (the specific DNA sequence defined therein being only an example). The polynucleotide sequence can be genomic DNA or cDNA. Polynucleotide sequences to which this invention relates also include those encoding substantially the same protein as that encoded by SEQ ID NO: 1, which include, for example, allelic forms of the given amino acid sequences and alternatively spliced products.

The present invention relates to a recombinant DNA molecule comprising a vector and a DNA sequence encoding BAEBL, or a portion thereof. Using methodology well known in the art, recombinant DNA molecules of the present invention can be constructed. Possible vectors for use in the present invention include expression vectors. The Plasmodium BAEBL encoding sequences of the present invention can be inserted into commercially available DNA vectors (expression vectors) to express the encoded protein product. The expression vectors have promoter sequences and other regulatory sequences necessary for expression in host cells. The technique of using expression vectors to introduce exogenous genes and express their protein products in a host cell is well known to those familiar with the art. For example the expression vector pET21a is commercially available and can be used to express proteins in E. coli. Alternatively the protein can be expressed in a eukaryotic cell, such as yeast, using Pichia expression vectors (i.e. pHIL-D2) commercially available from Invitrogen. The baculovirus system is also commercially available and can be used to express the BAEBL genes in insect cultures.

Once the baebl gene or fragment thereof has been cloned into an expression vector, the resulting vector can be used to transform a host cell, using procedures known to those familiar with the art. Such transformation procedures include but are not limited to microinjection, microprojectile bombardment, electroporation, calcium chloride permeabilization, polyethylene glycol permeabilization, protoplast fusion or bacterial mediated mechanisms such as Agrobacterium tumafaciens or Agrobacterium rhizogenes.

Host cells may be selected from any cell in which expression of modified proteins can be made compatible, including bacteria, fungus, yeast, plant cells and animal cells. Suitable host cells include prokaryotes selected from the genus Escherichia or Staphylococcus and eukaryotes selected from the genus Pichia (including Saccharomyces cerevisae, for example). In addition, mammalian cell culture (such as CHO and COS cells) can be used to express the BAEBL proteins and peptide fragments.

The transformed host cells synthesize the BAEBL protein or peptide fragment which can be isolated and purified using standard methods known to those familiar with the art. In one embodiment the BAEBL proteins and peptide fragments can be expressed as fusion proteins to assist in the purification of the BAEBL protein products.

The present invention also relates to a Plasmodium BAEBL protein separated from those proteins with which it is naturally associated. One skilled in the art can easily purify BAEBL using methodologies well known in the art.

The present invention further relates to a recombinantly produced BAEBL protein with the amino acid sequence given in SEQ ID NO: 2, an allelic variation thereof or a chimeric protein thereof. The present invention also relates to recombinantly produced peptide fragments of BAEBL. Further, the present invention relates to synthetic BAEBL or a synthetic peptide fragment thereof.

The present invention further relates to a polypeptide comprising an amino acid sequence having a consecutive number of amino acid sequences selected from a BAEBL protein, for example, Plasmodium BAEBL protein having the sequence of SEQ ID NO: 2, which are useful as diagnostic agents or can be utilized as therapeutic agents for treating or preventing malaria. Some agents are useful as antigenic fragments to display linear epitopes, and other agents are useful as antigenic fragments to display conformational epitopes for generating neutralizing antibodies. In another embodiment, the invention relates to a polypeptide comprising an amino acid sequence that encodes an EBA-175-like domain of a BAEBL protein, which is BAEBL region II, constituting two Duffy binding-like (DBL) domains, F1 and F2, for example, Plasmodium BAEBL protein having the sequence of SEQ ID NO: 2 running from amino acids 154-738. In another embodiment, the invention is directed to a polypeptide comprising an amino acid sequence having the following number of consecutive amino acids taken from a BAEBL protein, for example, Plasmodium BAEBL protein having the sequence of SEQ ID NO: 2: 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, and 584.

The present invention further relates to antibodies specific for epitopes present on the BAEBL proteins of the Plasmodium parasites. Thus, in one embodiment, the present invention relates to antibodies (such as monoclonal, polyclonal, chimeric, humanized, and anti-idiotypic) specific for the BAEBL protein. More particularly, the antibodies are directed to conserved regions of BAEBL and more preferably to BAEBL region II. One skilled in the art, using standard techniques well known to those skilled in the art can raise antibodies to the proteins and peptide fragments disclosed in the present invention. These antibodies can be useful as diagnostic agents or can be utilized as therapeutic agents for treating or preventing malaria.

The present invention relates to a vaccine for use in humans against malaria. As is customary for vaccines, BAEBL or a portion thereof, can be delivered to a human in a pharmacologically acceptable vehicle. As one skilled in the art will understand, it is not necessary to use the entire protein. A portion of the protein (for example, a peptide corresponding to a conserved region of the BAEBL protein) can be conjugated to pharmacologically acceptable carriers, including diphtheria toxoid, pertussis toxoid, or tetanus toxoid.

Vaccines of the present invention can include effective amounts of immunological adjuvants known to enhance an immune response. Adjuvants suitable for co-administration in accordance with the present invention should be ones that are potentially safe, well tolerated and effective in people. Such immunological adjuvants include QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59 (see Kim et al. 2000 Vaccine, 18: 597 and references therein).

The protein or peptide fragment is present in the vaccine in an amount sufficient to induce an immune response against the antigenic portion and thus to protect against Plasmodium infection thereby protecting the human against malaria. Protective antibodies are usually best elicited by a series of 2-3 doses given about 2 to 3 weeks apart. The series can be repeated when concentrations of circulating antibodies in the human drops. Further, the vaccine can be used to immunize a human against other forms of malaria (that is, heterologous immunization).

Compositions comprising substantially purified polynucleotide sequences encoding a BAEBL protein or peptide fragment can be used in accordance with the present invention as vaccines. Live vector viruses are contemplated, where retroviruses, adenoviruses, or adeno-associated viruses are engineered to carry polynucleotide sequences encoding a BAEBL protein or peptide fragment. Genetic immunization is an alternative, where naked DNA encoding a BAEBL protein or peptide fragment is administered to cells and the encoded protein antigens are expressed.

The present invention further yet relates to receptor blocking therapy which disrupts the life cycle of the parasite in humans. Administering to a human antibodies of the present invention specific for the binding site of the BAEBL ligand of the present invention can prevent invasion of red blood cells by the merozoite, a necessary event in the life cycle of the Plasmodium parasite. Alternatively, the erythrocyte receptor can be administered to a human, which is glycophorin C/D (Reid and Spring, 1994 Transfusion Medicine 4: 139). The BAEBL ligand on the merozoite will bind the circulating receptor rather than the determinate on the red blood cells. Attachment of the merozoite to the red blood cells, and hence invasion of the parasite, is prevented.

The major human malaria parasite, P. falciparum, has redundant or alternate receptor-ligand pathways of invasion. Therefore, an effective vaccine for blocking parasite invasion of erythrocytes by P. falciparum malaria will also target the redundant receptor ligand interactions that occur during the invasion process. Thus in some embodiments, the present vaccine compositions comprise a BAEBL polypeptide in combination with additional Plasmodium specific proteins or peptide fragments. For example, the second polypeptide may comprise an amino acid sequence that encodes a Duffy binding protein or erythrocyte binding antigen-175 (EBA-175) of a malaria Plasmodium parasite. The Duffy binding protein and EBA-175 are members of the EBL family of proteins that are utilized by Plasmodium parasites to invade erythrocytes. Thus, one vaccine composition in accordance with the present invention comprises two or more proteins (or peptide fragments) and a pharmaceutically acceptable vehicle, wherein at least one protein (or peptide fragment) is Duffy binding protein or EBA-175. EBA-175 and Duffy binding proteins of Plasmodium parasites have been described in the prior art as well as their use in preparing vaccines to prevent malaria infections. See U.S. Pat Nos. 5,198,347 and 6,120,770.

The present invention also relates to a method of vaccinating a vertebrate species, particularly a human, against a malaria Plasmodium parasite. The method comprises the steps of administering a vaccine composition comprising a protein or peptide fragment of BAEBL where the peptide fragment comprises at least a consecutive six amino acid sequence and a physiologically acceptable vehicle. In one embodiment the vaccine composition further comprises a second protein or peptide fragment wherein the second protein or peptide fragment comprises the Duffy binding protein or erythrocyte binding antigen-175 of an erythrocyte binding protein. The vaccine composition also can include various adjuvants known to those skilled in the art. The vaccine composition can be administered to a vertebrate species either orally or parenterally using techniques well known to those skilled in the art.

Nucleic Acid Molecules

As indicated herein, nucleic acid molecules of the present invention may be in the form of RNA or in the form of DNA obtained by cloning or produced synthetically. The DNA may be double-stranded or single-stranded. Single-stranded DNA or RNA may be the coding strand, also known as the sense strand, or it may be the non-coding strand, also referred to as the anti-sense strand.

By “isolated” nucleic acid molecule(s) is intended a nucleic acid molecule, DNA or RNA, which has been removed from its native environment. For example, recombinant DNA molecules contained in a vector are considered isolated for the purposes of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. Isolated nucleic acid molecules according to the present invention further include such molecules produced synthetically.

Nucleic acid molecules of the present invention include DNA molecules comprising an open reading frame (ORF) of a wild-type baebl gene; and DNA molecules which comprise a sequence substantially different from those described above but which, due to the degeneracy of the genetic code, still encode an ORF of a wild-type BAEBL polypeptide. Of course, the genetic code is well known in the art. Degenerate variants optimized for human codon usage are preferred.

In another aspect, the invention provides a nucleic acid molecule comprising a polynucleotide which hybridizes under stringent hybridization conditions to a portion of the polynucleotide in a nucleic acid molecule of the invention described above. By “stringent hybridization conditions” is intended overnight incubation at 42° C. in a solution comprising: 50% formamide, 5 times SSC (750 mM NaCl, 75 mM trisodium citrate), 50 mM sodium phosphate (pH 7.6), 5 times Denhardt's solution, 10% dextran sulfate, and 20 μg/ml denatured, sheared salmon sperm DNA, followed by washing the filters in 0.1 times SSC at about 65 degree C.

By a polynucleotide which hybridizes to a “portion” of a polynucleotide is intended a polynucleotide (either DNA or RNA) hybridizing to at least about 15 nucleotides (nt), and more preferably at least about 20 nt, still more preferably at least about 30 nt, and even more preferably about 30-70 nt of the reference polynucleotide.

By a portion of a polynucleotide of “at least 20 nt in length,” for example, is intended 20 or more contiguous nucleotides from the nucleotide sequence of the reference polynucleotide. Of course, a polynucleotide which hybridizes only to a complementary stretch of T (or U) resides, would not be included in a polynucleotide of the invention used to hybridize to a portion of a nucleic acid of the invention, since such a polynucleotide would hybridize to any nucleic acid molecule containing a poly T (or U) stretch or the complement thereof (e.g., practically any double-stranded DNA clone).

As indicated herein, nucleic acid molecules of the present invention which encode a BAEBL polypeptide may include, but are not limited to those encoding the amino acid sequence of the full-length polypeptide, by itself, the coding sequence for the full-length polypeptide and additional sequences, such as those encoding a leader or secretory sequence, such as a pre-, or pro- or prepro-protein sequence, the coding sequence of the full-length polypeptide, with or without the aforementioned additional coding sequences, together with additional, non-coding sequences, including for example, but not limited to introns and non-coding 5′ and 3′ sequences, such as the transcribed, non-translated sequences that play a role in transcription, mRNA processing, including splicing and polyadenylation signals, for example, ribosome binding and stability of mRNA; and additional coding sequence which codes for additional amino acids, such as those which provide additional functionalities.

The present invention further relates to variants of the nucleic acid molecules of the present invention, which encode portions, analogs or derivatives of the BAEBL protein. Variants may occur naturally, such as a natural allelic variant. By an “allelic variant” is intended one of several alternate forms of a gene occupying a given locus on a genome of an organism (Genes II, 1985 Lewin, B., ed., John Wiley & Sons, New York). Non-naturally occurring variants may be produced using art-known mutagenesis techniques.

Such variants include those produced by nucleotide substitutions, deletions or additions, which may involve one or more nucleotides. The variants may be altered in coding regions, non-coding regions, or both. Alterations in the coding regions may produce conservative or non-conservative amino acid substitutions, deletions or additions. Especially preferred among these are silent substitutions, additions and deletions, which do not alter the properties and activities of the BAEBL polypeptide or portions thereof Also especially preferred in this regard are conservative substitutions.

Further embodiments of the invention include nucleic acid molecules comprising a polynucleotide having a nucleotide sequence at least 70% identical, and more preferably at least 80%, 90%, 95% or 99% identical to a nucleotide sequence encoding a polypeptide having the amino acid sequence of a wild-type BAEBL polypeptide or a nucleotide sequence complementary thereto.

By a polynucleotide having a nucleotide sequence at least, for example, 95% “identical” to a reference nucleotide sequence encoding a BAEBL polypeptide is intended that the nucleotide sequence of the polynucleotide is identical to the reference sequence except that the polynucleotide sequence may include up to five point mutations per each 100 nucleotides of the reference nucleotide sequence encoding the BAEBL polypeptide. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence.

As a practical matter, whether any particular nucleic acid molecule is at least 70%, 80%, 90%, 95% or 99% identical to the reference nucleotide sequence can be determined conventionally using known computer programs such as the Bestfit program (Wisconsin Sequence Analysis Package, Version 8 for Unix, Genetics Computer Group, University Research Park, 575 Science Drive, Madison, Wis. 53711). Bestfit uses the local homology algorithm of Smith and Waterman, Advances in Applied Mathematics 2: 482-489 (1981), to find the best segment of homology between two sequences. When using Bestfit or any other sequence alignment program to determine whether a particular sequence is, for instance, 95% identical to a reference sequence according to the present invention, the parameters are set, of course, such that the percentage of identity is calculated over the full length of the reference nucleotide sequence and that gaps in homology of up to 5% of the total number of nucleotides in the reference sequence are allowed.

The present application is directed to nucleic acid molecules at least 70%, 80%, 90%, 95% or 99% identical to the nucleic acid sequences shown herein in the Sequence Listing which encode a polypeptide having BAEBL polypeptide activity. By “a polypeptide having BAEBL activity” is intended polypeptides exhibiting BAEBL activity in a particular biological assay. For example, BAEBL protein activity can be measured for changes in character by an appropriate erythrocyte binding assay.

Of course, due to the degeneracy of the genetic code, one of ordinary skill in the art will immediately recognize that a large number of the nucleic acid molecules having a sequence at least 70%, 80%, 90%, 95%, or 99% identical to a nucleic acid sequence shown herein in the Sequence Listing will encode a polypeptide “having BAEBL polypeptide activity.” In fact, since degenerate variants of these nucleotide sequences all encode the same polypeptide, this will be clear to the skilled artisan even without performing the above described comparison assay. It will be further recognized in the art that, for such nucleic acid molecules that are not degenerate variants, a reasonable number will also encode a polypeptide having BAEBL polypeptide activity. This is because the skilled artisan is fully aware of amino acid substitutions that are either less likely or not likely to significantly effect protein function (e.g., replacing one aliphatic amino acid with a second aliphatic amino acid).

For example, guidance concerning how to make phenotypically silent amino acid substitutions is provided in (Bowie, J. U. et al. 1990 Science 247: 1306-1310), wherein the authors indicate that proteins are surprisingly tolerant of amino acid substitutions.

Polypeptides and Fragments

The invention further provides a BAEBL polypeptide having the amino acid sequence encoded by an open reading frame (ORF) of a wild-type BAEBL gene, or a peptide or polypeptide comprising a portion thereof (e.g., region II).

It will be recognized in the art that some amino acid sequences of the BAEBL polypeptides can be varied without significant effect of the structure or function of the protein. If such differences in sequence are contemplated, it should be remembered that there will be critical areas on the protein which determine activity.

Thus, the invention further includes variations of the BAEBL polypeptide which show substantial BAEBL polypeptide activity or which include regions of BAEBL protein such as the protein portions discussed below. Such mutants include deletions, insertions, inversions, repeats, and type substitutions. As indicated, guidance concerning which amino acid changes are likely to be phenotypically silent can be found in (Bowie, J. U. et al. 1990 Science 247: 1306-1310).

Thus, the fragment, derivative or analog of the polypeptide of the invention may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which additional amino acids are fused to the mature polypeptide, such as a fusion peptide or leader or secretory sequence or a sequence which is employed for purification of the mature polypeptide or a proprotein sequence. Such fragments, derivatives and analogs are deemed to be within the scope of those skilled in the art from the teachings herein.

As indicated, changes are preferably of a minor nature, such as conservative amino acid substitutions that do not significantly affect the folding or activity of the protein (see Table A).

TABLE A
Conservative Amino Acid Substitutions

| | |
|---|---|
| Aromatic | Phenylalanine |
| | Tryptophan |
| | Tyrosine |
| Ionizable: Acidic | Aspartic Acid |
| | Glutamic Acid |
| Ionizable: Basic | Arginine |
| | Histidine |
| | Lysine |
| Nonionizable Polar | Asparagine |
| | Glutamine |
| | Serine |
| | Threonine |
| Nonpolar | Alanine |
| (Hydrophobic) | Glycine |
| | Isoleucine |
| | Leucine |
| | Proline |
| | Valine |
| Sulfur Containing | Cysteine |
| | Methionine |

Amino acids in the BAEBL polypeptides of the present invention that are essential for function can be identified by methods known in the art, such as site-directed mutagenesis or alanine-scanning mutagenesis (Cunningham and Wells, 1989 Science 244: 1081-1085). The latter procedure introduces single alanine mutations at every residue in the molecule. The resulting mutant molecules are then tested for biological activity such as changes in erythrocyte binding character.

The polypeptides of the present invention are conveniently provided in an isolated form. By “isolated polypeptide” is intended a polypeptide removed from its native environment. Thus, a polypeptide produced and/or contained within a recombinant host cell is considered isolated for purposes of the present invention.

Also intended as an “isolated polypeptide” are polypeptides that have been purified, partially or substantially, from a recombinant host cell or a native source. For example, a recombinantly produced version of the BAEBL polypeptide can be substantially purified by the one-step method described in Smith and Johnson, 1988 Gene 67: 31-40.

The polypeptides of the present invention include a polypeptide comprising a polypeptide shown herein in the Sequence Listing; as well as polypeptides which are at least 70% identical, and more preferably at least 80%, 90%, 95% or 99% identical to those described above and also include portions of such polypeptides.

By a polypeptide having an amino acid sequence at least, for example, 95% “identical” to a reference amino acid sequence of an BAEBL polypeptide is intended that the amino acid sequence of the polypeptide is identical to the reference sequence except that the polypeptide sequence may include up to five amino acid alterations per each 100 amino acids of the reference amino acid of the BAEBL polypeptide. In other words, to obtain a polypeptide having an amino acid sequence at least 95% identical to a reference amino acid sequence, up to 5% of the amino acid residues in the reference sequence may be deleted or substituted with another amino acid, or a number of amino acids up to 5% of the total amino acid residues in the reference sequence may be inserted into the reference sequence. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 95%, 96%, 97%, 98% or 99% identical to, for instance, the amino acid sequence shown herein in the Sequence Listing can be determined conventionally using known computer programs such the Bestfit program (Wisconsin Sequence Analysis Package, Version 8 for Unix, Genetics Computer Group, University Research Park, 575 Science Drive, Madison, Wis. 53711). When using Bestfit or any other sequence alignment program to determine whether a particular sequence is, for instance, 95% identical to a reference sequence according to the present invention, the parameters are set, of course, such that the percentage of identity is calculated over the full length of the reference amino acid sequence and that gaps in homology of up to 5% of the total number of amino acid residues in the reference sequence are allowed.

Pharmaceutical Formulations and Modes of Administration

The compounds of this invention can be employed in admixture with conventional excipients, i.e., pharmaceutically acceptable organic or inorganic carrier substances suitable for parenteral, enteral (e.g., oral) or topical application which do not deleteriously react with the active compounds. Suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. The pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. They can also be combined where desired with other active agents, e.g., vitamins.

For parenteral application, which includes intramuscular, intradermal, subcutaneous, intranasal, intracapsular, intraspinal, intrasternal, and intravenous injection, particularly suitable are injectable, sterile solutions, preferably oily or aqueous solutions, as well as suspensions, emulsions, or implants, including suppositories. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

For enteral application, particularly suitable are tablets, dragees, liquids, drops, suppositories, or capsules. The pharmaceutical compositions may be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. A syrup, elixir, or the like can be used wherein a sweetened vehicle is employed.

Sustained or directed release compositions can be formulated, e.g., liposomes or those wherein the active compound is protected with differentially degradable coatings, e.g., by microencapsulation, multiple coatings, etc. It is also possible to freeze dry the new compounds and use the lyophilizates obtained, for example, for the preparation of products for injection.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

For topical application, there are employed as non-sprayable forms, viscous to semi-solid or solid forms comprising a carrier compatible with topical application and having a dynamic viscosity preferably greater than water. Suitable formulations include but are not limited to solutions, suspensions, emulsions, creams, ointments, powders, liniments, salves, aerosols, etc., which are, if desired, sterilized or mixed with auxiliary agents, e.g., preservatives, stabilizers, wetting agents, buffers or salts for influencing osmotic pressure, etc. For topical application, also suitable are sprayable aerosol preparations wherein the active ingredient, preferably in combination with a solid or liquid inert carrier material, is packaged in a squeeze bottle or in admixture with a pressurized volatile, normally gaseous propellant, e.g., a freon.

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Reduced Binding to Gerbich Erythrocytes.

A new gene (baebl) of the DBL-EBP family of Plasmodium receptor proteins with erythrocyte specificity different from that of EBA-175 has been studied in P. falciparum. The exon/intron structure of BAEBL is similar to that of other DBL-EBL (Adams, J. H. et al. 1992 PNAS USA 89: 7085-7089). The difference between two DBL-EBP from P. falciparum and P. vivax/P. knowlesi is the duplication of region II (Adams, J. H. et al. 1992 PNAS USA 89: 7085-7089). Despite the similarity of BAEBL and EBA-175 in their requirement for sialic acid on erythrocyte proteins, the specificity of BAEBL for receptors on the erythrocyte surface differs from that of EBA-175. The differences are two-fold. First, BAEBL, but not EBA-175, binds to En(a-) erythrocytes that lack glycophorin A. Second, Gerbich erythrocytes (that have an altered glycophorin C and absent glycophorin D) bind BAEBL much more weakly than normal erythrocytes, but bind EBA-175 normally. Thus, the specificity of these two parasite receptor molecules differs, suggesting alternative pathways for invasion. These data indicate at least two different sialic acid-dependent pathways for invasion.

In preliminary experiments, BAEBL was not detected in the parasite supernatant absorbed with glycophorin C/D null erythrocytes of the Leach phenotype.

BAEBL, however, was never detected in the eluate of these erythrocytes. In addition, 2 and 8 μl of Leach erythrocytes, but not normal erythrocytes, removed BAEBL from parasite supernatant. The failure to elute BAEBL and its reduction with small numbers of Leach erythrocytes suggest proteolysis.

The Gerbich phenotype is found at high allele frequencies (50%) in some regions of Papua New Guinea (Booth, P. B. et al. 1982 Hum Hered 32: 385-403). The mutation consists of a deletion of exon 3 in glycophorin C that leads to truncated glycophorin C and absent glycophorin D (Serjeantson, S. W. et al. 1994 Immunol Cell Biol 72: 23-27). Of interest to our study is the fact that these areas co-localize to hyperendemic areas of malaria. Previously, Serjeantson (Serjeantson, S. W. 1989 Papua New Guinea Med J 32: 5-9) found reduced frequency of heavy infections with P. falciparum and P. vivax with the Gerbich phenotype. The common mutation in erythrocyte band 3, resulting in ovalocytosis, was not described at the time of the study by Serjeantson (Serjeantson, S. W. 1989 Papua New Guinea Med J 32: 5-9) and may have influenced the results. Pasvol et al. (Pasvol, G. et al. Lancet Apr. 21, 1984 907-908) found reduced invasion of Gerbich erythrocytes, but these were also ovalocytic (Aanstee, D. J. et al. 1984 Biochem J 218: 615-619). It is possible that hereditary ovalocytosis caused by band 3 mutations was influencing the invasion and frequency of infection. It is critical to restudy these groups now that the two mutations can be separated by molecular techniques. We were unable to find any reduction in invasion of Gerbich erythrocytes by the two P. falciparum clones, Dd2 and Dd2/Nm, but other parasite clones could be affected. It is known that Dd2 switched to the sialic acid-independent pathway (Dd2/Nm) after modification of the eba-175 gene (Dolan, S. A. et al. 1990 J Clin Invest 86: 618-624), suggesting that Dd2 lacking EBA-175 could not invade using BAEBL. Alternatively, the effect on invasion may be too subtle to be detected by the invasion assay as performed.

The tantalizing possibility remains that the high frequency of the Gerbich phenotype is selected for as a result of reduced invasion by P. falciparum mediated by the BAEBL receptor. The Gerbich mutation, like Duffy negativity in West Africa, appears to be going to fixation in these communities. The difference between the Gerbich mutation and Duffy negativity is that the Gerbich negative erythrocytes are still infected by P. falciparum while Duffy negative erythrocytes are refractory to P. vivax. Still, the parasite receptors for these two blood groups are envisioned as being immunogens to prevent malaria because mutation in the host molecules leads to no infection (P. vivax and Duffy negativity) or reduced infection (P. falciparum and Gerbich mutation).

Structure of the baebl Gene.

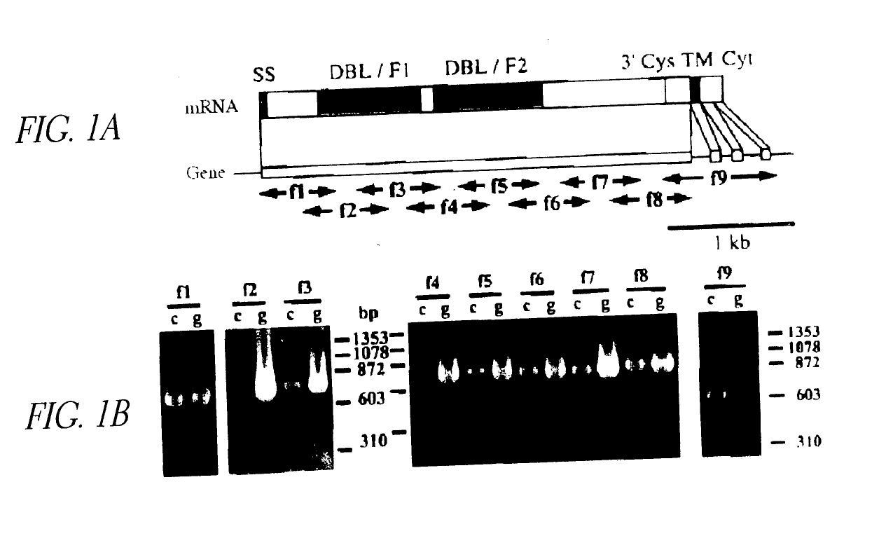

BAEBL is predicted to have two cysteine-rich domains (regions II and VI), a transmembrane region, and a cytoplasmic region. This structure is characteristic of all DBL-EBP genes (Adams, J. H. et al. 1992 PNAS USA 89: 7085-7089). The sequence was obtained from the Sanger Centre chromosome 13 genomic sequence of P. falciparum clone 3D7 (http://www.sanger.ac.uk/Projects/P_{—}falciparum/). The sequence of the gene was also determined from genomic and cDNA sequences of Dd2/Nm. The exon/intron structure for Dd2/Nm was identical to that of EBA-175 in that it had four exons: one for the extracellular domain, one for the transmembrane domain, and two encoding the cytoplasmic region (FIG. 1). The extracellular exon 1 of Dd2/Nm was identical to the genomic sequence of 3D7 from the Sanger Centre except for three changes in region II (I185V, N239S, and K261T; Dd2/Nm amino acid number from GenBank: AF332918/3D7 Sanger Centre chromosome 13). Using microsatellites and a genetic cross between Dd2 and HB3 (Su, X. et al. 1999 Science 286: 1351-1353), baebl was localized to the end of chromosome 13 close to marker C13M51.

Localization and Expression of BAEBL.

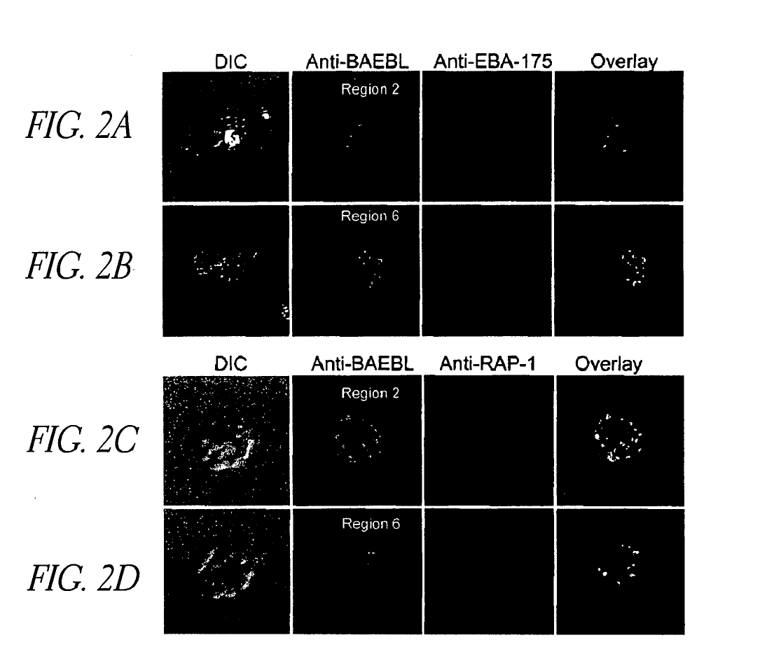

Antibodies to the two cysteine-rich domains (regions II and VI) for BAEBL of Dd2/Nm were used to determine localization and expression of the protein. Antibodies to regions II and VI localize to the same organelle as EBA-175 (FIG. 2A, B), which was previously shown to localize to the micronemes (Sim, B. K. L. et al. 1992 Mol Biochem Parasitol 51: 157-160). Furthermore, immunolocalization of RAP-1, a protein found in rhoptries, another apical organelle of merozoites, shows that BAEBL is adjacent to but not overlapping RAP-1 (FIG. 2C, D). This distribution is consistent with the localization of BAEBL within micronemes, a distribution identical to EBA-175 and the P. knowlesi Duffy binding protein (Adams, J. H. et al. 1990 Cell 63: 142-153; Sim, B. K. L. et al. 1992 Mol Biochem Parasitol 51: 157-160). The fact that antisera against two different regions of BAEBL showed identical localization within the parasite indicate that the antisera are not cross-reacting with another protein.

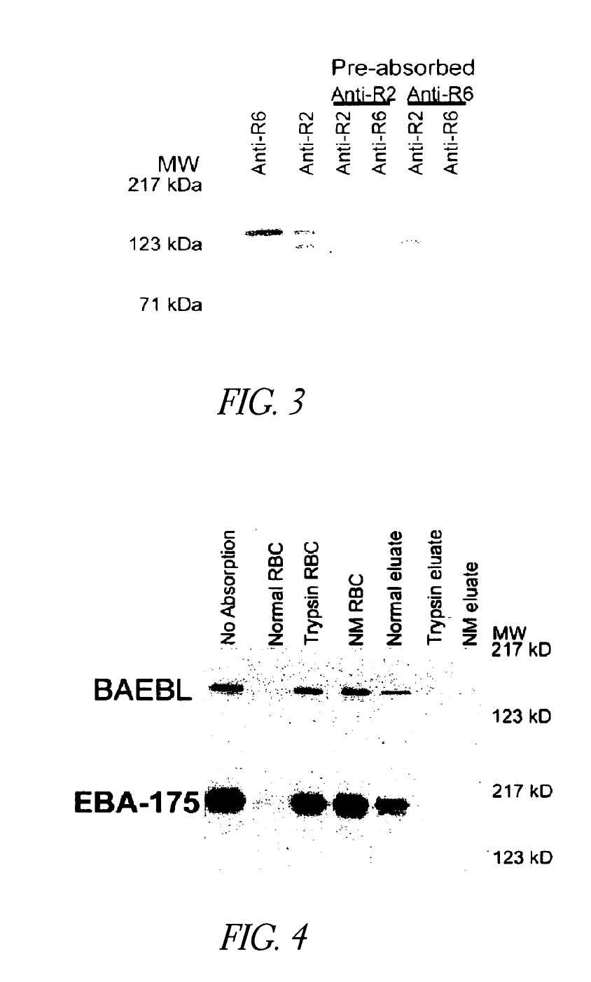

To study the molecular characteristics of BAEBL, we used methods developed for the production of soluble, metabolically labeled erythrocyte binding proteins (see Examples). Antisera to regions II and VI immunoprecipitated a protein of approximately 148 kDa; antibodies to region II also immunoprecipitated two lower molecular weight proteins (129 kDa and 117 kDa). The proof that the two 135-kDa proteins were identical derived from studies of immunoabsorption with one sera followed by immunoprecipitation with the second sera (FIG. 3). The same 135-kDa protein was removed by both sera, indicating that the antisera to the two regions of BAEBL were not cross-reacting with another protein. The two lower molecular weight proteins identified by anti-region II, but not by anti-region VI, resulted from immunoprecipitation of proteolytic products of BAEBL that contained region II but not region VI.

Erythrocyte Binding Specificity.

We have developed a new assay for measuring binding of BAEBL to erythrocytes. Previously, EBA-175 was identified in parasite proteins bound and eluted from human erythrocytes. Its identification was based on the fact that it was the most abundant and the highest molecular weight protein eluted from these erythrocytes. Lower molecular weight proteins may be proteolytic fragments of EBA-175 or products of different genes. To positively identify BAEBL, we immunoprecipitated BAEBL from proteins eluted from erythrocytes with anti-region II and anti-region VI.

We also modified the protocol in that we identified proteins removed from the supernatant by erythrocyte absorption. We determined the different quantities of erythrocytes required to remove BAEBL from the parasite supernatant. It was found that 25 μl of packed erythrocytes slightly reduced the quantity of immunoprecipitated BAEBL from 50 μl of parasite supernatant. The protein was largely removed by absorbing twice with 50 μl of packed erythrocytes for some culture supernatants and by only one absorbtion with 50 μl for other supernatants. Therefore, on some samples, we absorbed with 25 μl, 50 μl once, and 50 μl twice for comparison between normal erythrocytes and mutant erythrocytes or enzyme-treated erythrocytes. The protein was also eluted from the first 50 μl of packed erythrocytes used for absorption. This set the conditions for absorbing and eluting BAEBL and demonstrated that BAEBL may be a parasite receptor for binding erythrocytes.

To determine the specificity of binding, we studied binding to neuraminidase-and trypsin-treated human erythrocytes and human erythrocytes with various genetically modified blood groups. Both enzymes eliminated the binding of BAEBL to human erythrocytes (FIG. 4), indicating that the erythrocyte receptor required sialic acid attached to a peptide backbone and must therefore be a sialoglycoprotein. This failure of neuraminidase-and trypsin-treated erythrocytes to bind was identical to EBA-175 (FIG. 4). To determine whether BAEBL was binding to the carbohydrates on the erythrocyte receptor, we performed competitive inhibition with Neu5Ac(α2-3) lactosialic and Neu5Ac(α2-6)lactosialic acid at 1 μM, 10 μM, 100 μM, and 1000 μM. We determined that neither Neu5Ac(α2-3) nor Neu5Ac(α2-6) lactosialic acid inhibited the binding of BAEBL to human erythrocytes. These results indicate that BAEBL is binding either to a more complex polysaccharide or to a combination of sialic acid and a peptide backbone of an erythrocyte sialoglycoprotein.

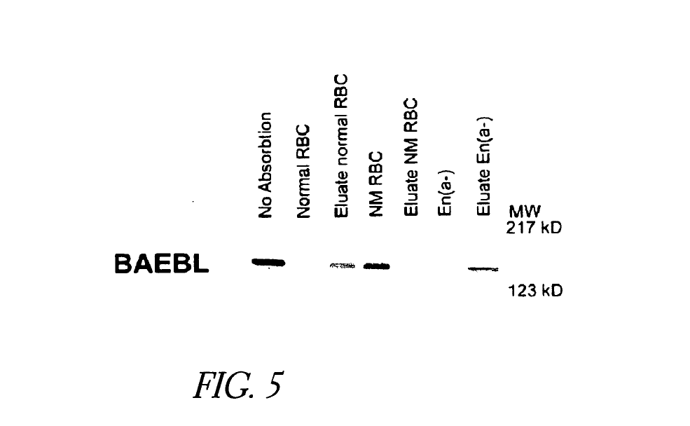

To further define the binding specificity of BAEBL, we studied En(a-) erythrocytes, which lack glycophorin A. We found that EBA-175 failed to bind En(a-) erythrocytes as previously described (Sim, B. K. L. et al. 1994 Science 264: 1941-1944). BAEBL, however, bound to these erythrocytes in a similar manner as to normal erythrocytes (FIG. 5). This demonstrated that the binding specificity of BAEBL differed from that of EBA-175. S-s-U-erythrocytes that lacked glycophorin B bound both EBA-175 and BAEBL. Thus, neither glycophorin A nor B is the sole receptor for BAEBL.

Abnormal Binding to Glycophorin C/D Mutant Erythrocytes.

Another characterized sialoglycoprotein on the surface of human erythrocytes is glycophorin C/D (Reid, M. E. & Spring, F. A. 1994 Transfusion Med 4: 139-146; Colin, Y. & Le Van Kim, C. 1995 in: Blood Cell Biochemistry, eds. Cartron, J. P. & Rouger, P. Plenum Press, New York pp. 331-350). Its peptide backbone is completely unrelated to glycophorins A and B but, like these, it has a mucin-like region of serines and threonines for O-linked sugars at the N-terminus of the protein. Both glycophorin C and glycophorin D are encoded by the same gene with use of alternative start codons. Glycophorin C, the full-length protein, contains one N-linked glycan. There are three mutations of the glycophorin C/D gene that lack high-incidence antigens (Colin, Y. & Le Van Kim, C. 1995 in: Blood Cell Biochemistry, eds. Cartron, J. P. & Rouger, P. Plenum Press, New York pp. 331-350). Leach erythrocytes are null for these proteins; Gerbich and Yus erythrocytes contain exon 3 and 2 deletions, respectively, that lead to a shortened glycophorin C and absent glycophorin D. Both Gerbich and Yus cells have abnormal N-linked glycosylation of the truncated form of glycophorin C (Reid, M. E. & Spring, F. A. 1994 Transfusion Med 4: 139-146).

We screened for the binding of BAEBL to erythrocytes of the Gerbich (-2, -3, -4) and Yus (-2, -3, -4) phenotype that had been frozen as pellets in liquid nitrogen. BAEBL had reduced binding to Gerbich and Yus erythrocytes. These differences were consistent for pellet-frozen erythrocytes from different donors. EBA-175 bound normally to these erythrocytes.

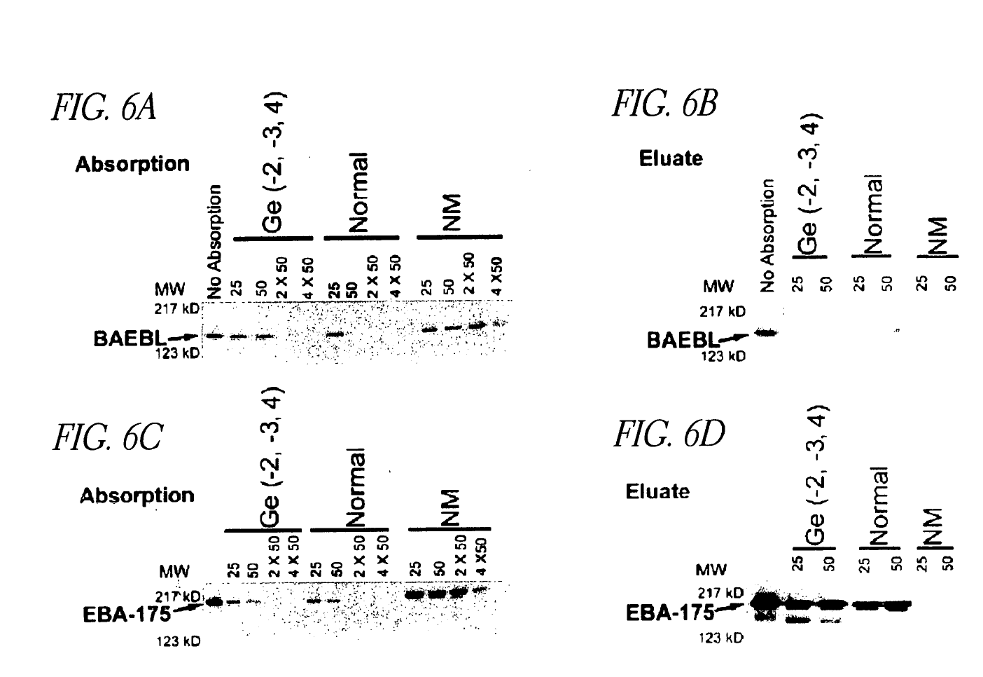

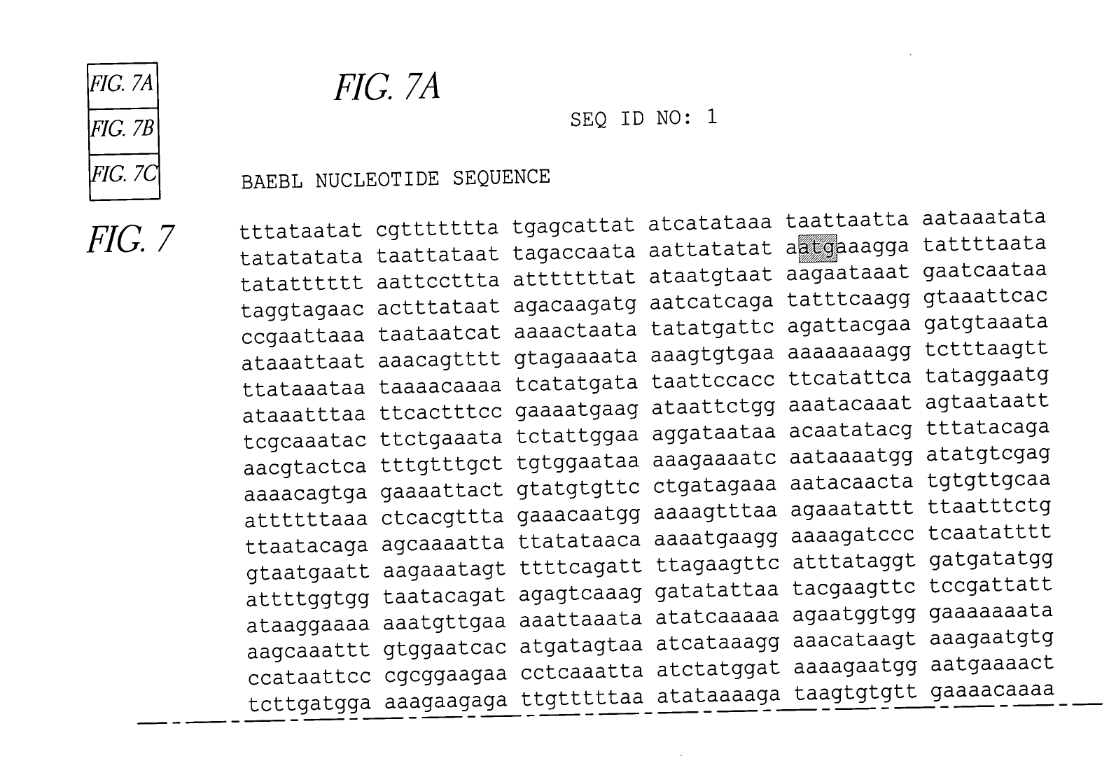

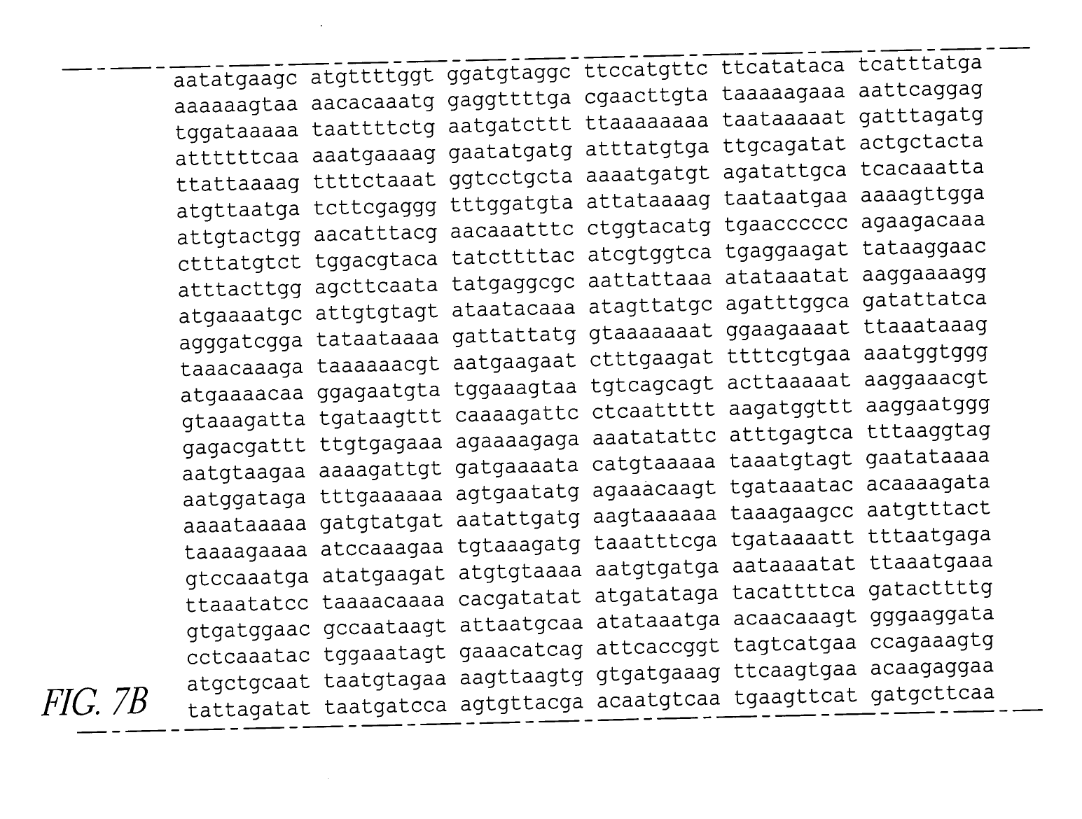

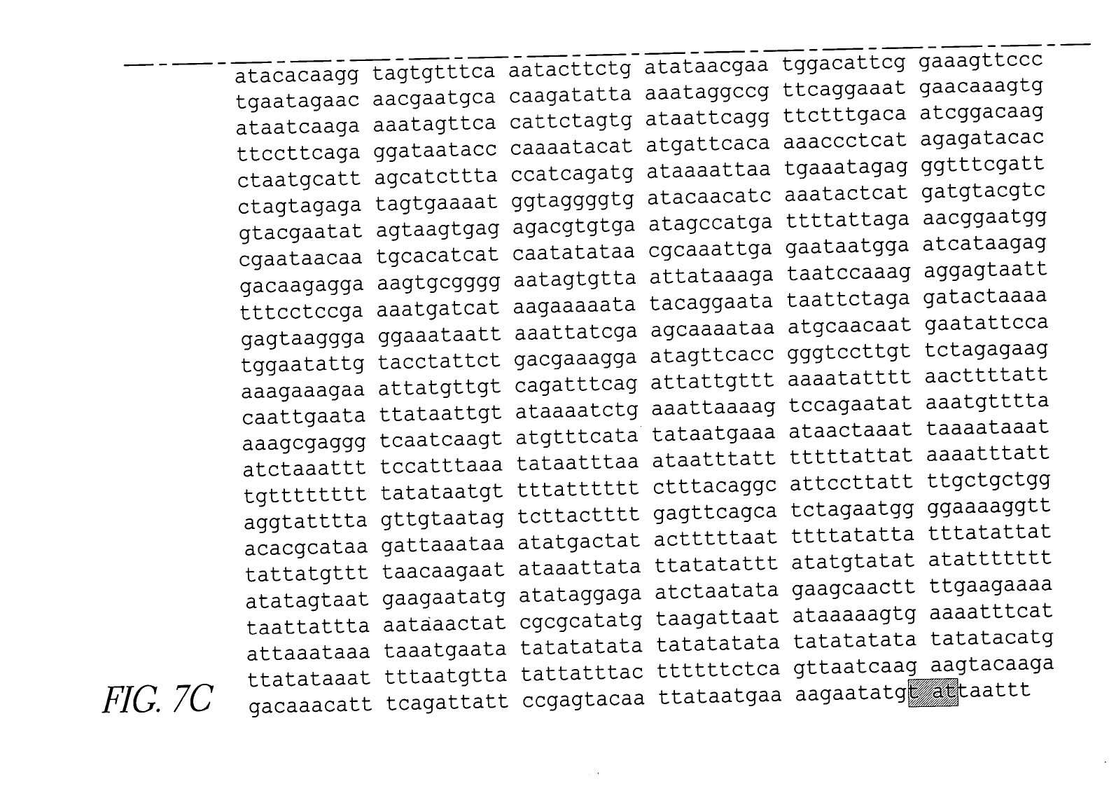

Because the quality of the pellet-frozen erythrocytes was unpredictable, we obtained fresh blood from a person with the Gerbich mutation. In two separate experiments, we found that it required twice as many Gerbich cells to remove BAEBL from the culture supernatant compared with normal erythrocytes (FIG. 6A). In contrast to BAEBL, EBA-175 bound equally well to Gerbich and normal erythrocytes (FIG. 6C). This difference between normal and Gerbich erythrocytes for absorption of BAEBL was similar to the results obtained with pellet-frozen erythrocytes as described above.

BAEBL was eluted from normal but not Gerbich erythrocytes, indicative of its poor binding to Gerbich erythrocytes (FIG. 6B). BAEBL also did not elute from neuraminidase-treated normal erythrocytes. These results are indicative of poor binding of BAEBL to Gerbich erythrocytes. In contrast to BAEBL, EBA-175 was eluted from both Gerbich and normal erythrocytes (FIG. 6D).

Invasion of Gerbich Erythrocytes.

P. falciparum clones Dd2 and Dd2/Nm invaded Gerbich erythrocytes at the same rate as normal erythrocytes (Table 1). Dd2 but not Dd2/Nm had markedly reduced invasion into neuraminidase-treated erythrocytes as described previously (Dolan, S. A. et al. 1990 J Clin Invest 86: 618-624).

TABLE 1
Invasion Rate of P. falciparum Into Gerbich Erythrocytes

P. falciparum clones

| Red-cell type | Dd2, % | Dd2/Nm, % |
|---|---|---|
| Normal | 3.7* | 1.6 |
| Gerbich | 3.0 | 1.8 |
| Neuraminidase-treated normal | 0 | 1.8 |
| Rhesus | 0 | 0 |

*Percentage of ring-infected erythrocytes.

### EXAMPLE 1

Structure of BAEBL.

The sequence of BAEBL was identified (Adams J. H., et al. 2001 Trends Parasitol 17: 17297-17299) from cDNA (GenBank No. N97830) deposited by D. Chakrabarti and from the database supplied by Sanger for chromosome 13 (>MAL13_{—}001500, Dec. 27, 2000). Based on this sequence, we sequenced the P. falciparum clone, Dd2/Nm (Dolan, S. A. et al. 1990 J Clin Invest 86: 618-624) from genomic DNA (GenBank No. AF332918). The exon/intron boundaries were defined by RT-PCR of the P. falciparum clone Dd2/Nm (GenBank No. AF332919 ). Primers use for Dd2/Nm sequencing were:

(SEQ ID NO: 3)

f1, 5′-AGACCAATAAATTATATATAATGAAAGGA-3′
and

(SEQ ID NO: 4)

5′-TTTAAACTTTTCCATTGTTTCTAAACG-3′;

(SEQ ID NO: 5)

f2, 5′-ATAAATTTAATTCACTTTCCGAAAATGA-3′
and

(SEQ ID NO: 6)

5′-AAAACAATCTCTTCTTTTCCATCAAG-3′;

(SEQ ID NO: 7)

F3, 5′-TTTATAGGTGATGATATGGATTTTGG-3′
and

(SEQ ID NO: 8)

5′-TCGTAAATGTTCCAGTACAATTCCT-3′;

(SEQ ID NO: 9)

f4, 5′-CAAATGGAGGTTTTGACGAACTTG-3′
and

(SEQ ID NO: 10)

5′-TAAGTACTGCTGACATTACTTTCCA-3′;

(SEQ ID NO: 11)

f5, 5′-GGAGCTTCAATATATGAGGCGCA-3′
and

(SEQ ID NO: 12)

5′-ATATCTTCATATTCATTTGGACTCTC-3′;

(SEQ ID NO: 13)

f6, 5′-TGAGTCATTTAAGGTAGAATGTAAGA-3′
and

(SEQ ID NO: 14)

5′-GGAACTTTCCGAATGTCCATTCGT-3′;

(SEQ ID NO: 15)

f7, 5′-TAAATGAACAACAAAGTGGGAAGGA-3′
and

(SEQ ID NO: 16)

5′-ATTCTCAATTTGCGTTATATATTGATG-3′;

(SEQ ID NO: 17)

f8, 5′-AGTTCCTTCAGAGGATAATACCCA-3′
and

(SEQ ID NO: 18)

5′-CTTGATTGACCCTCGCTTTTAAAAC-3′;

(SEQ ID NO: 19)

f9, 5′-ACTAAAAGAGTAAGGGAGGAAATAAT-3′
and

(SEQ ID NO: 20)

5′-TATAAAATACATTGAATTATTTAAACTATTG-3′.

PCR from total RNA untreated with reverse transcriptase never produced PCR-amplified products. Oligonucleotides 5′-ATTCCTTATTTTGCTGCTGGAGGT-3′ (SEQ ID NO: 21) and 5′-AAGTTGCTTCTATATTAGATTCTCCT-3′ (SEQ ID NO: 22) were also used to sequence fragment f9. Only the 3′-region was sequenced for cDNA to determine the precise location of the intron/exon boundaries.

Antisera.

Antisera to BAEBL region II and region VI of Dd2/Nm were generated by immunization of rats with a DNA vaccine using the vector VR1050 (kindly supplied by Stephen Hoffman, Naval Medical Research Center, Silver Spring, Md.) that contains the T cell epitopes P2P30 from tetanus toxoid. Region II and region VI gene fragments of BAEBL were amplified from P. falciparum clone Dd2/Nm and cloned into VR1050 vector, previously described as VR1012tPAp2p30 by Becker et al. (Becker, S. I. et al. 1998 Infect Immun 66: 3457-3461) but now renamed VR1050. The inserts for regions II and VI of Dd2/Nm spanned from amino acids Q141 to I756 and K1046 to S1132, respectively (GenBank No. AF332918). Rats were immunized intradermally with 500 μg of DNA at 3-week intervals for a total of four immunizations. Sera were obtained from the rats a week after the fourth immunization.

Rabbit anti-region II of EBA-175 (KLS14) was a kind gift of David Narum and Kim Lee Sim (EntreMed, Rockville, Md.). Mouse anti-RAP-1 monoclonal antibody 7H8/50 [MRA-79, Malaria Research and Reference Reagent Resource (MR4) Center] was a kind gift of Allan Saul (Queensland Institute of Medical Research, Brisbane, Australia).

Erythrocytes Used in the Studies.

Blood was collected in 10% citrate-phosphate-dextrose (vol/vol) and stored for up to 4 weeks at 4° C. At the time of study, the erythrocytes were washed three times in incomplete media (RPMI-1640; Life Technologies, Rockville, Md.) with 25 mM HEPES and 0.36 mM hypoxanthine (Sigma, St. Louis, Mo.). For neuraminidase treatment, 5.5 ml of a 5% (vol/vol) suspension of the washed human erythrocytes in incomplete media were incubated twice with 3 milliunits of neuraminidase (Vibrio cholerae; CalBiochem, La Jolla, Calif.) for 2 hr at 37° C. each time. For trypsin treatment, washed human erythrocytes were incubated with 1 mg/ml of tosyl-phenylalanine-chloromethyl-ketone-treated trypsin (Sigma) for 2 hr at 37° C. After trypsin treatment, the cells were washed once in incomplete medium and incubated with 2 mg/ml soybean trypsin inhibitor (Sigma) for 10 min at room temperature. The cells were washed twice before use in a study.

The glycophorin A and glycophorin B null erythrocytes [En(a-) and S-s-U-, respectively and the glycophorin D null/glycophorin C modified erythrocytes (Gerbich cells) were frozen within a few days of receipt and thawed by the Red Cross method (Mallory, D. ed. 1993 Immunohematology Methods and Procedures American Red Cross, National Reference Laboratory, Rockville, Md., pp. 125-1-125-2). Blood from a Gerbich donor was collected in 10% (vol/vol) anticoagulant citrate-phosphate-dextrose.

Other glycophorin C/D mutant cells (Leach, Gerbich, and Yus cells) had been stored in liquid nitrogen as frozen pellets (Judd, W. J. 1994 in: Methods in Immunohematology Montgomery Scientific Publications Durham, N.C., pp. 188-190) and thawed directly into PBS at 37° C.

Metabolic Labeling of Parasite Proteins.

Soluble, metabolically labeled parasite proteins were obtained from culture supernatant of schizont-infected erythrocytes that released merozoites in the absence of uninfected erythrocytes. The parasites were left to lyse and release proteins into the culture supernatant. The Dd2/Nm clone of P. falciparum was cultured as previously described (Kaneko, O. et al. 2000 Mol Biochem Parasitol 110: 135-14) with the following exceptions. Schizont-infected erythrocytes (5×10⁷ per ml of culture medium) were used during the metabolic labeling. The culture supernatant was ultracentrifuged in a Beckman Optima TLX Ultracentrifuge (Beckman, Fullerton, Calif.) at 40,000 rpm (98,600×g) for 10 min at 4° C. before storage at −70° C.

Immunoprecipitation.

Proteins in the supernatant and in the diluted eluate were immunoprecipitated as previously described (Kaneko, O. et al. 2000 Mol Biochem Parasitol 110: 135-146) with the following exceptions. The supernatant (50 μl) was diluted into 250 μl of NETT (50 mM Tris, pH 7.4, 150 mM NaCl, 1 mM EDTA, and 0.5% Triton X-100) supplemented with 0.5% bovine serum albumin (BSA; ICN, Aurora, Ohio). To determine whether the proteins immunoprecipitated by anti-BAEBL region II and anti-BAEBL region VI are identical, we preabsorbed with one antisera and immunoprecipitated with the other. Radiolabeled supernatant (50 μl) was preabsorbed with protein A-Sepharose as previously described (Kaneko, O. et al. 2000 Mol Biochem Parasitol 110: 135-146). The supernatant was incubated with 10 μl of anti-BAEBL region II or 10 μl of anti-BAEBL region VI for 2 hr at 4° C. Protein G-Sepharose (40 μl; 50% vol/vol) was added to remove the immune complexes. Supernatant was split into two equal volumes and immunoprecipitated with 5 μl of anti-BAEBL region II and 5 μl anti-BAEBL region VI as described above.

Modified Erythrocyte Binding Assay.

Erythrocyte binding assays were developed for metabolically labeled proteins (as described above) that bind erythrocytes. The original assay required that parasite proteins be bound and eluted from some erythrocytes and not from others. In the original study (Camus, D. & Hadley, T. H. 1985 Science 230: 553-556), the major protein eluted from the erythrocytes was EBA-175. Lower molecular proteins could be proteolytic fragments of EBA-175 or other proteins. Furthermore, this assay is insensitive for less abundant proteins. Therefore, we have developed a new assay that depends on the identification of BAEBL with two antisera against different regions of BAEBL and its removal from the culture supernatant by human erythrocytes. The parasite protein can also be identified and quantified by elution of bound protein from erythrocytes followed by immunoprecipitation. It is then possible to study its specificity for erythrocyte receptors with erythrocytes lacking various proteins or with enzymatically modified erythrocytes. First, we determined the quantity of erythrocytes that would remove the majority of BAEBL and used this quantity with erythrocytes of various types (enzyme-modified erythrocytes and erythrocytes genetically deficient in membrane proteins) to determine the erythrocyte specificity of BAEBL. We found that one or two absorptions with a volume of packed erythrocytes equal to the volume of metabolically labeled supernatant were required to remove BAEBL from the supernatant, depending on the concentration of BAEBL in the supernatant.

Elution from erythrocytes of bound parasite proteins was performed as described previously (Kaneko, O. et al. 2000 Mol Biochem Parasitol 110: 135-146). Parasite proteins were eluted only from the erythrocytes of the first adsorption. The parasite proteins were eluted as previously described. Because of the adverse effect of high salt on immunoprecipitation, the eluate was diluted 5 fold (vol/vol) in NETT with 0.5% BSA prior to immunoprecipitation.

Competitive Inhibition Assay.

An inhibition assay was conducted in the presence of Neu5Ac(α2-3) lactosialic acid or Neu5Ac(α2-6) lactosialic acid (Sigma). Metabolically labeled parasite supernatant (50 μl) was preincubated with 1, 10, 100, or 1000 μM in 15 μl of the aforementioned carbohydrates for 1 hr at room temperature. Packed erythrocytes (50 μl) were added to the mixture. The erythrocyte binding assay was conducted as described above. Immunolocalization of BAEBL.

The methods for immunolocalization of BAEBL by confocal microscopy were performed as previously described (Kaneko, O. et al. 2000 Mol Biochem Parasitol 110: 135-146) with the following modifications. The blocking buffer consisted of PBS (pH 7.4) containing 0.1% Triton X-100 (Bio-Rad, Hercules, Calif.) and 2.5 mg/ml normal goat serum (Jackson ImmunoResearch Laboratories, West Grove, Pa.). The secondary antisera consisted of Alexa 488-conjugated goat anti-rat IgG and Alexa 594-conjugated goat anti-rabbit IgG (Molecular Probes, Eugene, Oreg.) diluted 1:500 in blocking buffer. For antiquenching, we mounted labeled parasites in Prolong Antifade (Molecular Probes).

Invasion Assay.

Pre-washed A⁺ human erythrocytes treated with neuraminidase and A⁺ human erythrocytes of the Gerbich type (-2, -3, -4) were tested for invasion by P. falciparum clones Dd2 and Dd2/Nm (Dolan, S. A., et al. 1990 J Clin Invest 86: 618-624) as described previously (Kaneko, O. et al. 1999 Exp Parasitol 93: 116-119). Rhesus (Macaca mulatta) erythrocytes that are resistant to invasion by P. falciparum were used as a control for normal erythrocytes introduced with the parasitized erythrocytes.

### EXAMPLE 2

Plasmodium falciparum has evolved great flexibility in its invasion pathways, in part as a result of multiple copies of the Duffy binding-like (DBL) family of erythrocyte-binding ligands, three of which have different red blood cell (RBC) receptor specificities (Sim, B. K. L. et al. 1994 Science 264: 1941-1944; Mayer D. C. G. et al. 2001 PNAS USA 98: 5222-5227). This is in contrast to P. vivax, which has a single copy of the DBL family member that recognizes the Duffy blood group antigen, explaining the resistance to P. vivax infection by humans who lack the Duffy blood group (Chitnis C. E. et al. 1994 J Exp Med 180: 497-506). The multiplicity of P. falciparum invasion pathways explains the lack of RBC refractory to invasion. We now describe another mechanism for recognition of different molecules on the RBC by P. falciparum, namely, amino acid polymorphisms in the DBL gene, BAEBL, that lead to different RBC specificities (Adams J. H., et al. 2001 Trends Parasitol 17: 17297-17299).

Initial characterization of RBC receptors recognized by BAEBL from three different P. falciparum clones in three different laboratories suggested that each had a different RBC receptor (Sim, B. K. L. et al. 1994 Science 264: 1941-1944; Thompson, J. K. et al. 2001 Mol Microbiol 41: 47-58; Narum, D. L. et al. 2002 Mol Biochem Parasitol 119: 159-168). We sequenced the baebl gene from eight parasite clones and found polymorphisms restricted to regions I and II of the molecules. In total, we sequenced region II of BAEBL from 11 clones from Papua New Guinea (PNG) and 13 clones from other parts of the world where P. falciparum malaria is highly prevalent. We observed five different sequence variants in region II with polymorphisms in four amino acid positions (Table 2). Unlike Africa where P. falciparum clones were introduced from Asia, PNG P. falciparum populations are isolated. Surprisingly, the same sequence variants occurred in PNG as in the rest of the world, suggesting that mutations leading to these polymorphisms occurred multiple times. Region II of P. falciparum DBL genes are duplicated forming the F1 and F2 domains (Adams J. H. et al. 1992 PNAS USA 89: 7085-7089). All base substitutions in the erythrocyte binding domain of BAEBL occurred in the F1 domain, whereas mutations in EBA-175, another DBL gene of P. falciparum, were scattered throughout both F1 and F2 domains and appear not to alter the RBC binding specificity (Liang, H. & Sim, B. K. 1997 Mol Biochem Parasitol 84: 241-245).

TABLE 2
Position of Polymorphisms in BAEBL From Different Malaria-Endemic Regions

| | | Region I* | | Region II* | | | |
|---|---|---|---|---|---|---|---|
| Clones | Origin | 26 | 112 | 185 | 239 | 261 | 285 |
| PNG2 | PNG† | I (ATT) | L(CTT) | V(GTT) | S(AGT) | T(ACG) | K(AAA) |
| PNG3 | PNG | I (ATT) | F(TTT) | I(ATT) | N(AAT) | R(AGG) | E(GAA) |
| PNG4 | PNG | I (ATT) | L(CTT) | V(GTT) | S(AGT) | K(AAG) | K(AAA) |
| E12 | PNG | I (ATT) | F(TTT) | I(ATT) | S(AGT) | K(AAG) | K(AAA) |
| 1917 | PNG | I (ATT) | L(CTT) | V(GTT) | S(AGT) | K(AAG) | K(AAA) |
| PNG13 | PNG | I (ATT) | F(TTT) | V(GTT) | S(AGT) | K(AAG) | K(AAA) |
| PNG5 | PNG | I (ATT) | L(CTT) | I(ATT) | S(AGT) | K(AAG) | K(AAA) |
| 1905 | PNG | I (ATT) | L(CTT) | V(GTT) | S(AGT) | K(AAG) | K(AAA) |
| PNG9-3 | PNG | I (ATT) | L(CTT) | V(GTT) | S(AGT) | K(AAG) | K(AAA) |
| PNG9-1 | PNG | I (ATT) | L(CTT) | V(GTT) | S(AGT) | T(ACG) | K(AAA) |
| PNG10-1 | PNG | I (ATT) | L(CTT) | I(ATT) | S(AGT) | K(AAG) | K(AAA) |
| M24 | Kenya | I (ATT) | F(TTT) | I(ATT) | N(AAT) | K(AAG) | K(AAA) |
| 3D7 | Africa? | I (ATT) | F(TTT) | I(ATT) | N(AAT) | K(AAG) | K(AAA) |
| Sc/d6 | Sierra Leone | I (ATT) | L(CTT) | V(GTT) | S(AGT) | K(AAG) | K(AAA) |
| Fab9 | Kwazulu | I (ATT) | L(CTT) | I(ATT) | N(AAT) | R(AGG) | E(GAA) |
| Dd2 | Vietnam | I (ATA) | L(CTT) | V(GTT) | S(AGT) | T(ACG) | K(AAA) |
| Camp | Malaysia | I (ATT) | L(CTT) | I(ATT) | N(AAT) | R(AGG) | E(GAA) |
| Dd2/Nm | Vietnam | I (ATT) | L(CTT) | V(GTT) | S(AGT) | T(ACG) | K(AAA) |
| T2/c6 | Thailand | I (ATT) | F(TTT) | I(ATT) | N(AAT) | K(AAG) | K(AAA) |
| MT/S-1 | Asia | I (ATT) | F(TTT) | I(ATT) | S(AGT) | K(AAG) | K(AAA) |
| HB3 | Honduras | I (ATT) | L(CTT) | V(GTT) | S(AGT) | K(AAG) | K(AAA) |
| PC49 | S. America | I (ATT) | F(TTT) | I(ATT) | N(AAT) | K(AAG) | K(AAA) |
| DIV30 | Brazil | I (ATT) | L(CTT) | V(GTT) | S(AGT) | T(ACG) | K(AAA) |
| PC26 | S. America | I (ATT) | L(CTT) | V(GTT) | S(AGT) | T(ACG) | K(AAA) |

*For Regions I and II, numbers refer to the amino acids in the sequence of BAEBL from GenBank AF332918. Mutated bases and amino acids are shown in bold.
†PNG, Papua New Guinea.

We investigated the functional significance of polymorphisms in the erythrocyte-binding domain of BAEBL. We expressed region II of four polymorphic groups transiently on the surface of COS cells with the T8 vector (Buffet P. A. et al. 1999 PNAS USA 96: 12743-12748). This was followed by an erythrocyte-binding assay as described previously (Chitnis C. E. et al. 1994 J Exp Med 180: 497-506). Binding was performed with normal and enzyme-treated (trypsin and neuraminidase) erythrocytes and Gerbich-negative erythrocytes (exon 3 deletion of glycophorin C/D) (Sim, B. K. L. et al. 1994 Science 264: 1941-1944; Mayer D. C. G. et al. 2001 PNAS USA 98: 5222-5227). Each of the polymorphisms led to a different binding specificity as demonstrated by different binding patterns to enzyme-treated and Gerbich-negative RBC (Table 3). Furthermore, a single base change led to a change in amino acid and RBC specificity (e.g., VSTK to VSKK). Such polymorphism in sequence and receptors was described for influenza hemagglutinin, where a single base mutation changed the amino acid and the specificity of binding to sialic acid (Rogers, G. N. et al. 1983 Nature 304: 76-78).

TABLE 3
Binding Patterns of BAEBL Variants to Enzyme-Treated
and Gerbich-Negative RBC

| Region II | Normal RBC | | | Gerbich-negative |
|---|---|---|---|---|
| variants* | Untreated^{†} | Trypsin^{‡} | Neuraminidase^{‡} | RBC^{‡} |
| VSTK | 65 | 0 | 0 | 0.9% |
| VSKK | 58 | 0 | 90% | 112% |
| ISKK | 59 | 92% | 0 | 96% |
| INRE | 67 | 114% | 110% | 100% |

*Region II of BAEBL expressed in COS cells is from amino acid 143 to 606 (GenBank AF332918) and contains the mutations shown at the positions delineated in Table 2.
^{†}COS cells with five or more attached RBC were counted and the total per coverslip recorded.
^{‡}Data from enzyme-treated and Gerbich-negative RBC are expressed as the percentage of binding to normal, untreated RBC.

The Gerbich-negative phenotype occurs at an allelic frequency of 50% in some regions of PNG (Booth, P. B. et al. 1982 Human Hered 32: 385-403). It is tantalizing to postulate that the polymorphisms in BAEBL could be a coevolutionary adaptation to the disappearance of the RBC receptor in Gerbich-negative individuals. Why then has the Gerbich-negative phenotype not been described in all the geographic areas where mutations in region II of BAEBL have occurred? One possibility is that the Gerbich-negative phenotype could indeed be more widespread than previously described. Alternatively, such polymorphisms in BAEBL receptor specificity may be advantageous to the parasite independently of the Gerbich negative phenotype.

While the present invention has been described in some detail for purposes of clarity and understanding, one skilled in the art will appreciate that various changes in form and detail can be made without departing from the true scope of the invention. All figures, tables, and appendices, as well as patents, applications, and publications, referred to above, are hereby incorporated by reference.

1. A vaccine composition comprising a polypeptide and a pharmaceutically acceptable vehicle, wherein the polypeptide comprises an amino acid sequence that encodes a BAEBL polypeptide or portion thereof.

2. A vaccine composition of claim 1, wherein the polypeptide portion is an amino acid sequence that encodes a BAEBL region II or portion thereof.

3. A vaccine composition of claim 2, wherein the polypeptide portion is selected from the group consisting of an amino acid sequence having the following number of consecutive amino acids taken from said BAEBL polypeptide:
6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, and 584.

4. A vaccine composition of any of claims 1-3 wherein said BAEBL polypeptide or portion thereof is defined as having the amino acid sequence of SEQ ID NO: 2 or portion thereof.

5. A vaccine composition of any of claims 1-3 wherein said BAEBL polypeptide or portion thereof is defined as having at least 70%, 80%, 90%, 95%, or 99% identity to the amino acid sequence of SEQ ID NO: 2 or portion thereof.

6. A vaccine composition of any of claims 1-3 wherein said BAEBL polypeptide or portion thereof is encoded by a polynucleotide defined as having at least 70%, 80%, 90%, 95%, or 99% identity to the open reading frame of SEQ ID NO: 1 or portion thereof.

7. A vaccine composition of any of claims 1-3 wherein said BAEBL polypeptide or portion thereof is encoded by a polynucleotide which hybridizes at 42 degree C. in a solution comprising: 50% formamide, 5 times SSC (750 mM NaCl, 75 mM trisodium citrate), 50 mM sodium phosphate (pH 7.6), 5 times Denhardt's solution, 10% dextran sulfate, and 20 μg/ml denatured, sheared salmon sperm DNA, followed by washing the filters in 0.1 times SSC at about 65 degree C., to a second polynucleotide having the polynucleotide sequence of SEQ ID NO: 1.

8. A vaccine composition of any of claims 1-3, wherein said BAEBL polypeptide or portion thereof has a polymorphism selected from the group consisting of I at position 185, N at position 239, T at position 261, R at position 261, and E at position 285.

9. A vaccine composition of any of claims 1-3 further comprising an adjuvant selected from the group consisting of QS-21, Detox-PC, MPL-SE, MoGM-CSF, TiterMax-G, CRL-1005, GERBU, TERamide, PSC97B, Adjumer, PG-026, GSK-1, GcMAF, B-alethine, MPC-026, Adjuvax, CpG ODN, Betafectin, Alum, and MF59.

10. A vaccine composition of any of claims 1-3 further comprising a second polypeptide, wherein said second polypeptide comprises an amino acid sequence that encodes at least a portion of a Duffy binding protein or erythrocyte binding antigen-175 (EBA-175) of a malaria Plasmodium parasite.

11. A vaccine composition comprising a polynucleotide and a pharmaceutically acceptable vehicle, wherein the polynucleotide comprises a nucleic acid sequence that encodes a BAEBL polypeptide or portion thereof.

12. A vaccine composition of claim 11, wherein the polypeptide portion is an amino acid sequence that encodes a BAEBL region II or portion thereof.

13. A vaccine composition of claim 12, wherein the polypeptide portion is selected from the group consisting of an amino acid sequence having the following number of consecutive amino acids taken from said BAEBL polypeptide:
6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, and 584.

14. A vaccine composition of any of claims 11-13 wherein said BAEBL polypeptide or portion thereof is defined as having the amino acid sequence of SEQ ID NO: 2 or portion thereof.

15. A vaccine composition of any of claims 11-13 wherein said BAEBL polypeptide or portion thereof is defined as having at least 70%, 80%, 90%, 95%, or 99% identity to the amino acid sequence of SEQ ID NO: 2 or portion thereof.

16. A vaccine composition of any of claims 11-13 wherein said BAEBL polypeptide or portion thereof is encoded by a polynucleotide which is identical to the open reading frame of SEQ ID NO: 1 or portion thereof.

17. A vaccine composition of any of claims 11-13 wherein said BAEBL polypeptide or portion thereof is encoded by a polynucleotide defined as having at least 70%, 80%, 90%, 95%, or 99% identity to the open reading frame of SEQ ID NO: 1 or portion thereof.

18. A vaccine composition of any of claims 11-13 wherein said BAEBL polypeptide or portion thereof is encoded by a polynucleotide which hybridizes at 42 degree C. in a solution comprising: 50% formamide, 5 times SSC (750 mM NaCl, 75 mM trisodium citrate), 50 mM sodium phosphate (pH 7.6), 5 times Denhardt's solution, 10% dextran sulfate, and 20 μg/ml denatured, sheared salmon sperm DNA, followed by washing the filters in 0.1 times SSC at about 65 degree C., to a second polynucleotide having the polynucleotide sequence of SEQ ID NO: 1.

19. A vaccine composition of any of claims 11-13, wherein said BAEBL polypeptide or portion thereof has a polymorphism selected from the group consisting of I at position 185, N at position 239, T at position 261, R at position 261, and E at position 285.

20. A method of vaccinating a human against a malaria Plasmodium parasite comprising the step of administering the vaccine composition of any of claims 1-3 or 11-13 to said human.

21. The method of claim 20 wherein said step of administration is by protein immunization.

22. The method of claim 20 wherein said step of administration is by genetic immunization.

23. A method of vaccinating a human against a malaria Plasmodium parasite comprising the step of administering antibodies specific for the binding site of a BAEBL ligand in an amount sufficient to inhibit the ligand from binding red blood cells in the human.
